(19) United States
(12) Patent Application Publication
(10) Pub. No.: US 2007/0275053 A1
(43) Pub. Date: Nov. 29, 2007

(54) Anti-stick formula delivered by spray process to facilitate swallowing of solid object, such as pill, tablet capsule or caplet.

(76) Inventor: Luise Anneliese Lenk, Tampa, FL (US)

Correspondence Address: Luise Anneliese Lenk, Suite 202, 5701 Mariner Street, Tampa, FL, 33609, US

(21) Appl. No.: 11/805,997

(22) Filed: May 26, 2007

Related U.S. Application Data
(60) Provisional application No. 60/803,321, filed on May 26, 2006.

(51) Int. Cl. A61K 9/28; A61K 9/48; A61K 9/20

(52) U.S. Cl. 424/451; 424/464; 427/2.14

(57) ABSTRACT

An oral delivery method and composition that will facilitate the swallowing of any object such as a pill, tablet, capsule or caplet in a fast, easy, untroubled manner so as to markedly decrease the risk of the pill, caplet, capsule or tablet lodging or sticking in the mouth or the throat. It is a process that involves spraying a specially formulated mixture onto both sides of the pill or other object so that it can be more easily swallowed. The mixture includes an anti-stick and lubricating agent, a natural flavoring and other agents so that the mixture is emulsified and can be easily digested. The mixture is placed in a plastic bottle and rendered into a mist through a high viscosity handheld pump. The mist thoroughly coats the pill, providing optimum lubrication for swallowing when accompanied by drinking water or another liquid.

I'm claiming the benefit of my earlier filed provisional patent application (Application No. 60/803,321, filed May 26, 2006).

### FIELD OF INVENTION

The present invention relates generally to a delivery system and method to facilitate pill swallowing, and more particularly to applying a lubricating, non-stick formula in spray form onto a pill, caplet, capsule, or tablet or other object to optimize swallowing.

### CROSS-REFERENCE TO RELATED APPLICATIONS

Not applicable

### REFERENCES SITED

### U.S. Patent Documents

| | | |
|---|---|---|
| 5,618,004 | April 1997 | Klearman et al. |
| 5,823,451 | October 1998 | Sharpe |
| 5,863,001 | January 1999 | Schulze |
| 5,915,637 | June 1999 | Parsons |
| 5,924,636 | July 1999 | Calderon |
| 6,059,209 | May 2000 | Barson |
| 2,436,505 | February 1948 | DuRall |
| 4,581,013 | April 1986 | Allen |
| 4,792,333 | December 1988 | Kidder |
| 5,643,204 | March 1996 | Cover |

### STATEMENT REGARDING FEDERALLY SPONSORED RESEARCH OR DEVELOPEMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

It is well known that many people have difficulty swallowing a pill, tablet, capsule or caplet. Research has shown nearly 60% of all seniors have had at least some trouble swallowing pills. In addition, it is estimated that 10% of young adults have difficulty swallowing.

The problem may be linked to inadequate saliva production, a growth in the throat area or anxiety about getting a pill stuck in the throat, which impedes the swallowing process. This problem is exacerbated by pills or other medications that are large, do not have a slick surface or must be broken in two, leaving a rough surface that can catch in the throat. The problem may also be caused by the chemical composition of the pill. According to Kikendall et al., Digestive Diseases and Sciences 28:2(1983), there were 221 cases documented between 1970 and 1982 of tablet- and capsule-induced esophageal injury. The most commonly implicated drugs were tetracycline (108 cases), emepronium bromide (36 cases), potassium chloride (16 cases) and ferrous salts (12 cases).

Swallowing is a complex process that occurs in a relatively brief period of time. When a pill or other solid object is swallowed, it must travel through an irregular channel as it works its way down the throat. Drinking water or another liquid with the pill is the common method to induce swallowing, but this may not be sufficient for some people or for some pills. There is a variety of other methods to improve pill swallowing. People can imbibe the pill with applesauce; eat ice cream or something else cold beforehand; place the pill at the back of the throat to induce the swallowing reflex; or stand up while swallowing the pill in hopes that gravity will pull it down. While these methods are helpful to some, they are ineffective or not feasible for others.

Manufacturers have tried to overcome difficulties of pill swallowing in many ways, also with incomplete success. Some medications have been reformulated into sprays and liquids, but many drugs cannot be dissolved in water because they would leave a very unpleasant taste when not encapsulated in a pill. Some medications are applied through the rectum, but this is not a popular option.

Pill splitters may sometimes help because a smaller object is easier to swallow, but, as previously mentioned, the rough edge of a split pill can catch in the throat and some pills, such as capsules, are not intended to be broken open.

Another attempt to aid swallowing involves crushing the pill. There are many patents with various designs for pill crushers, such as U.S. Pat. Nos. 5,618,004 to Klearman et al., Apr. 8, 1997; 5,823,451 to Sharpe, Oct. 20, 1998; 5,863,001 to Schulze, Jan. 26, 1999; 5,915,637 to Parsons, Jun. 29, 1999; 5,924,636 to Calderon, Jul. 20, 1999, and 6,059,209 to Barson, May 9, 2000. Many pills, however, should not be crushed because the mixture underneath the coating has an unpleasant taste, may irritate the lining of the mouth or may contain a sustained-release agent that must start at certain sites in the body, such as the intestine.

Other patents are meant to facilitate swallowing of an uncrushed pill, usually by using tubes that combine liquid with the pill. Some examples:

U.S. Pat. No. 2,436,505 to DuRall describes a pill douser for administering medicines in pills or tablets as well as in liquid form. A tube draws liquid from a drinking glass and combines it with the medicine, which sits in a bowl.

U.S. Pat. No. 4,581,013 to Allen describes a doser for orally administering a solid medication. A tube with a removable closure and a radically extending plate supports the medication and permits passage of a stream of liquid.

U.S. Pat. No. 4,792,333 to Kidder describes a package for orally administering a solid substance. A tube has two portions that support and confine the substance but permit fluid flow.

Meanwhile, U.S. Pat. No. 5,643,204 to Cover describes an intra-oral device to facilitate pill swallowing using a shield that covers part of the roof of the mouth.

All these processes and devices appear to be cumbersome and it is not clear that they always function well or are comfortable to use.

In contrast, spraying a unique lubricating, anti-stick formula onto pills and then swallowing them with water is simple to understand, thus making it more likely to be used. Unlike the previously mentioned inventions, it is not uncomfortable and can be employed without complications.

### BRIEF SUMMARY OF THE INVENTION

The present invention specifically addresses the problems, identified above, with previous methods and inventions that try to facilitate pill swallowing. More specifically, the present invention employs a device that sprays a non-stick, formula onto both sides of a pill or other object so that it can be more easily swallowed. The device is a plastic bottle with a hand-held, high viscosity pressure sprayer that turns liquid formula in the bottle into a concentrated mist.

The liquid formula includes Medium Chain Triglycerides, an anti-stick and lubricating agent. Even though MCT is thicker than water, its viscosity is low enough so that it can still be sprayed into a mist, coating the object to be swallowed. MCT can be easily metabolized by the liver. In addition to MCT, the formula contains a number of liquid vitamin supplements and natural flavoring, which improve lubrication and provide a pleasant taste, thus making it more likely that this process will be used.

### BRIEF DESCRIPTION OF THE DRAWINGS

The accompanying drawings illustrate the invention. In such drawings:

FIG. 1 is a perspective view illustrating the process that is used to spray the anti-stick, lubricating formula onto a pill, tablet, capsule or caplet.

FIG. 2 shows the anti-stick, lubricating effect on a pill or other solid object as it passes through the mouth and throat to the stomach in the act of swallowing with water or any other liquid.

### DETAILED DESCRIPTION OF THE PREFERRED EMBODIMENT

The detailed description set forth below in connection with the appended drawings is intended as a description of the presently preferred embodiments of the invention, and is not intended to represent the only forms in which the present invention may be constructed or utilized. The description sets forth the functions and sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

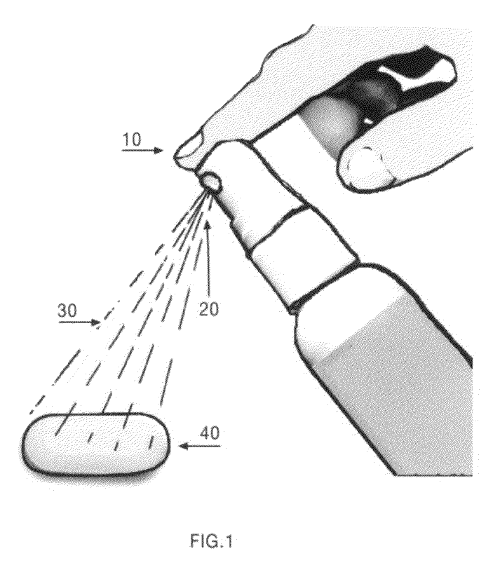

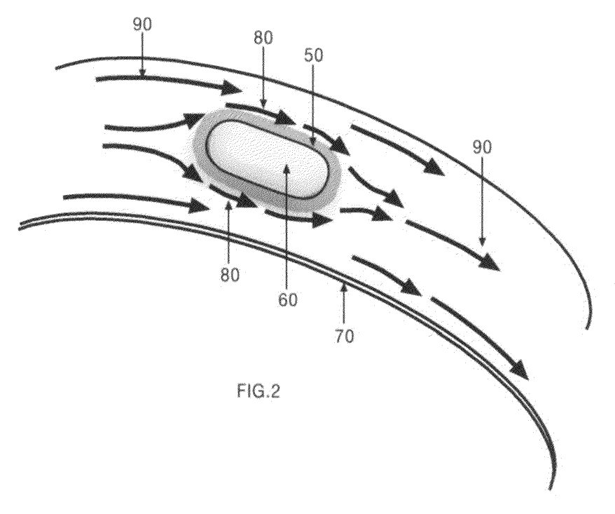

The pill-swallowing process, using a liquid formula and spraying device, is illustrated in FIGS. 1 and 2.

Referring to FIG. 1, Finger (10) presses high viscosity hand pump (20), spraying the misted formula over the pill, tablet, capsule or caplet (40). It is the Applicant's contention that once the pill is coated with the anti-stick formula (30), swallowing the pill coated with the formula along with any liquid is much more effective than swallowing the pill with the liquid alone.

Continuing to FIG. 2, the anti-stick, lubricating formula (50) creates an effective layer around the pill or other solid object (60) that serves to ensure that the pill does not stick to the surface of the tongue, roof of the mouth. esophagus or throat passage (70) and continues to move in the direction of flow (80) to the stomach along with the liquid (90) that is carrying the pill.

The key element that allows for this process to work is Medium Chain Triglycerides (MCTs) that are in the liquid formula. MCT oil is a clear liquid with no flavor. It has been chosen specifically for its relatively low viscosity, at approximately 25 millipascal seconds (mPa·s), which permits the formula to convert to a fine mist when sprayed through a high viscosity handheld pump. In comparison, some other oils have higher viscosities, such as canola oil at 57 mPa s. Oils like this cannot be converted into an even spray. For example, when olive oil is sprayed, it leaves a messy, irregular coating.

In addition to its functional viscosity, MCTs are more easily digested and absorbed by the body than are conventional fats. MCT oil does not go into the lymphatic system but is transported directly to the liver, where it is metabolized. Medium-Chain Triglycerides consist of a mixture of triglycerides of saturated fatty acids, mainly of caprylic acid and capric acid. The fatty acids are derived from the oil extracted from the hard, dried fraction of the endosperm of Cocos nucifera L. or from the dried endosperm of Elaeis guineensis Jacq. Due to their low viscosity and their oxidative stability, MCTs are also used as excipients in oral, rectal and topical pharmaceuticals. Due to their smaller molecular size, MCTs are digested much more rapidly than are long chain triglycerides.

Most naturally occurring fats are triglycerides; the “tri” means that each molecule has three carbon chains. Medium chain triglycerides have chains made up of six to 12 carbon atoms. MCT oils occur naturally and the most abundant source is coconut oil. When MCT oil is metabolized in the body, it behaves more like a carbohydrate, which means that it will be used as energy rather than be stored as fat.

The formula also contains Vitamin E (_-tocopherol). Antioxidants such as vitamin E act to protect the body's cells against the effects of free radicals, which are potentially damaging by-products of energy metabolism. Free radicals can damage cells and may contribute to the development of cardiovascular disease and cancer.

The mixture also contains flavoring to add a pleasant taste and eliminate bitter pill aftertaste, thus facilitating swallowing. It is currently formulated in wintergreen and cherry flavors, although other flavors may be added at a later date.

The bottle is made of polyethylene terephthalate (PET) plastic, a thermoplastic polymer resin of the polyester family. It is lightweight, shatter-resistant and, when correctly manufactured, does not leak or absorb the liquid it contains.

The high viscosity sprayer (either hand pump or mechanical) makes a fine mist of the formula, even though the formula has a higher viscosity than water. The mist permits ease of swallowing. It allows for total coverage of the pill, without soaking it, as would occur if it were dipped into the liquid.

While the invention has been described with reference to a preferred embodiment, it is clearly understood that the invention is not limited thereto. For example, while wintergreen and natural cherry are currently the preferred flavors, other flavors could easily be added or used. Thus, the scope of the invention is to be interpreted only in conjunction with the described claims.

Using the product with anti-stick and lubricating properties delivered from a hand-held bottle with a fine mist sprayer, when applied on both sides of any solid object such as a pill, tablet, capsule or caplet, will coat them in such a way that, when taken with any liquid, facilitates swallowing of said pill, tablet, capsule, caplet or other object.

A formula that, when applied through the process of spraying, on any solid object such as a pill, tablet, capsule, caplet or other object and then taken with water or any liquid will markedly decrease the risk of the pill, tablet capsule; caplet or other object becoming lodged on the tongue or palate, or in the throat or esophagus, in comparison to taking the pill, tablet, capsule, caplet or other object while simply imbibing water or another liquid.

What is claimed specifically is:

1) A process to apply a lubricating, anti-stick formula onto pills, tablets, capsules, caplets or other objects to facilitate swallowing of said pill, tablet, capsule, caplet or other object.

2) A process of claim 1 wherein the anti-stick formula is applied from a hand-operated bottle with a fine-mist spray nozzle.

3) A process of claim 2 wherein using a high-efficiency handheld pump distributes the formula evenly over the pill, tablet, capsule, caplet or other object.

4) A process of claim 1 where the anti-stick formula is applied using a mechanically generated spray.

5) A process of claim 4 wherein using a high-efficiency mechanical pump distributes the formula evenly over the pill tablet, capsule, caplet or other object.

6) A lubricating, anti-stick formula wherein the principle component of the lubricating formula is an oil.

7) A formula of claim 6 wherein the oil is preferably an oil comprising fatty acid glyceride esters that are liquid at room temperature.

8) A formula of claim 7 wherein the oil component comprises Medium Chain Triglycerides (MCTs).

9) A formula of claim 8 wherein the viscosity is preferably less than 50 mPa·s

10) A formula of claim 8 wherein the viscosity is preferably in the range 20-30 mPa·s

11) A formula of claim 8 wherein the MCTs are triglyceride esters comprising mainly caprylic (C:8) and capric (C:10) fatty acids.

12) A formula of claim 11, wherein the lubricating, anti-stick formula contains a natural or synthetic flavoring.

13) A formula of claim 12 wherein the flavoring improves palatability of the coated pill, tablet, capsule, caplet or other object.

14) A formula of claim 12 wherein the flavoring facilitates swallowing by providing a pleasant taste.

15) A formula of claim 12 wherein the flavoring comprises less than 10% of the total formula by volume.

16) A formula of claim 12 wherein the flavoring comprises preferably less than 5% of the total formula by volume.

17) A formula of claim 12 wherein the flavoring comprises wintergreen, peppermint, spearmint, lemon, orange, lime, anise, caraway, cherry, grape, vanilla or any other flavoring that might increase palatability and aid swallowing of the pill, tablet, capsule, caplet or other object.

18) A formula of claim 17, wherein the lubricating, anti-stick formula contains an antioxidant

19) A formula of claim 18, wherein the antioxidant comprises vitamin (_-tocopherol).

20) A formula of claim 18 wherein the vitamin E is present as either _-tocopherol, _-tocopherol acetate, _-tocopherol succinate or _-tocopherol polyethylene glycol 1000 succinate.

21) A formula of claim 18 wherein the _-tocopherol comprises less than 0.1% w/v in the total formulation adjusting for the Vitamin E activity of the different forms.

22) A formula of claim 18 wherein the _-tocopherol comprises less than 0.05% w/v in the total formulation adjusting for the Vitamin E activity of the different forms.
